(19) United States
(12) Patent Application Publication
(10) Pub. No.: US 2018/0252686 A1
(43) Pub. Date: Sep. 6, 2018

(54) Vortical Thin Film Reactor

(71) Applicant: DAVID A. SMITH, Lawrenceville, NJ (US)

(72) Inventors: Michael C. Trachtenberg, Lawrenceville, NJ (US); David A. Smith, Lawrenceville, NJ (US)

(21) Appl. No.: 15/911,111

(22) Filed: Mar. 3, 2018

Related U.S. Application Data
(60) Provisional application No. 62/467,132, filed on Mar. 5, 2017.

(51) Int. Cl. G01N 30/42; B01D 11/04; B04B 5/04; A61M 1/36

(52) U.S. Cl. CPC G01N 30/42; B01D 11/048; A61M 1/3696; B04B 5/0428; B04B 5/0442

(57) ABSTRACT

We describe vortical thin layer film flow along a spiral channel designed to improve mass and heat transfer efficiency for a multitude of physicochemical reactions and processes. Spiral channels, commonly augmented by centrifugal rotation, support rapid reaction between one or more fluids in a given channel. Dean vortices generate screw-shaped patterns processing axially in the channel, repeatedly refreshing radial interfaces. Fluids self-align, self-assemble, stable, controllable, exhibit thin film geometry. Multiple discrete lamellae can flow with independent velocity separated by density and may be soluble or insoluble in one another. Membranes separating spirals allow other interactions. Energy can be provided and extracted from each flow. Flows can enter or exit independently along the channel length. The pressure within each channel is controlled even when operated at the liquid's vapor pressure. The device is scalable to include a multiplicity of flows in a multiplicity of centrifugally rotating chambers.

### CROSS REFERENCE RELATED APPLICATIONS

N/A

### FEDERAL SPONSORSHIP

N/A

### REFERENCES CITED

### Patents

Allen R. W. K., MacInnes J. M., Priestman G. H. 2006. EP1703970 A1. Fluid contactor.

Ito Y. 1982. U.S. Pat. No. 4,321,138. Method and apparatus for preparative countercurrent chromatography employing a rotating column assembly.

Ito Y. 1984. U.S. Pat. No. 4,425,112. Flow through centrifuge.

Ito Y. 1995. U.S. Pat. No. 5,449,461. Displacement countercurrent chromatography.

Ito Y. 2002. U.S. Pat. No. 6,379.973 B1. Chromatographic separation apparatus and method.

Yu, H. Z., Parameswaren, M., Li, P. C. H., Peng, X. Y., Chen, H., Chou, W. L. 2013. U.S. Pat. No. 8,343,788 B2. Microfluidic microarray assembles and methods of manufacturing and using.

### OTHER REFERENCES

Hatzell K. B., Beidaghi M, Campos J. W., Dennison C. R., Kumbur E. C., Gogotsi Y. 2013. A high performance pseudocapacitive suspension electrode for the electrochemical flow capacitor. Electrochim Acta. 111:888-897.

Hornung C. H. and Mackley M. R. 2009. The measurement and characterization of residence time distributions for laminar liquid flow in plastic microcapillary arrays. Chem. Eng. Sci. 64:3389-3902.

Ito Y. 2000. Centrifugal precipitation chromatography: principle, apparatus, and optimization of key parameters for protein fractionation by ammonium sulfate precipitation. Anal. Biochem. 277:143-153.

Jeon S-I., Park H-R., Yeo J-G., Yang S. C., Cho C. H., Han M. H., Kim D. K. 2013. Desalination via a new membrane capacitive deionization process utilizing flow-electrodes. Energy Environ. Sci., 6:1471-1475.

MacInnes J. M., Ayash A. A., Dowson G. R. M. 2017. CO2 absorption using diethanolamine-water solutions in a rotating spiral contactor. Chem. Eng. J. 307:1084-1091.

MacInnes J. M., Ortiz-Osorio J., Jordan P. J., Priestman G. H., Allen R. W. K. 2010. Experimental demonstration of rotating spiral microchannel distillation. Chem. Eng. J. 159:159-169.

MacInnes, J. M., Pitt M. J., Priestman G. H., Allen R. W. K. 2012. Analysis of two-phase contacting in a rotating spiral channel. Chem. Eng. Sci. 69:304:315.

MacInnes J. M. and Zambri M. K. S. 2015. Hydrodynamic characteristics of a rotating spiral fluid-phase contactor. Chem. Eng. Sci. 126:427-439.

Oka, H., Harada, K-I., Nakazawa, H, Ito, Y. 1989. Foam counter-current chromatography of bacitracin I. Batch separation with nitrogen and water free of additives. J. Chromatogr. 482:197-205.

Peng, X. Y. and Li, P. C. H. 2008. Centrifugal pumping in the equiforce spiral microchannel. Can. J. Pure Appl. Sci. 2:551-556.

Yang Y., Aisa H. A., Ito Y. 2009. Mathematical model of computer-programmed intermittent dual countercurrent chromatography applied to hydrostatic and hydrodynamic equilibrium systems. J. Chromatogr. A. 1216:6310-6318.

Zambri M. K. S. 2014. Rotating spiral prototype contactor: Design and demonstration. Thesis. Univ. of Sheffield, UK

### BACKGROUND

### Field of the Invention

The present invention is in the technical field of apparatus and processes to enable and enhance physical and chemical processes, physicochemical separations and reactions and apparatus to enable such processes. More specifically, it is in the field of efficiency enhancement of mass and heat transfer as applied to separations and reactions with special reference to mass and heat transfer efficiency by means of thin film, laminar flow interfacial reactions.

### Description of the Prior Art

High efficiency chemical and physicochemical processes require optimization of mass and heat transfer, as well as controlled reaction processes to minimize secondary purification and limit wastage. Reviews on the subject routinely lament these limitations in currently available equipment and processes. The more important contributing factors to achieve increased efficiency and better overall performance via optimization of mass and heat transfer are laminar flow, interfacial replenishment, a large, stable interfacial area, optimization of flow reactivity under defined conditions and the ability to effect local and global temporal and spatial catalysis and defined process activation.

The most common approaches towards achieving these goals focus on the use of membrane separators, microfluidic flow, and process intensification. In recent years MacInnes and his colleagues have evaluated these approaches and laid out strong cases concerning their limitations [2010, 2012, 2014, 2015, 2017]. All of these arguments are incorporated by reference. Their concerns as well as others from the literature lead to the following conclusions:

Membranes present a complex tradeoff as they involve additional cost for materials, assembly and replacement, diffusional flow limitations and pressure drop as well as fouling and failing. Other limits include complexity and high operating energy costs. On the other hand, scale-up is far more easily achieved when membranes separate adjacent fluids as path length is limited only by pump size. Membranes provide size or ion selectivity, for example. They are the heart of plate and frame designs. Another limitation of membranes is poor mass or heat transfer. Further, membranes are generally used in rectilinear format whether the device has that form or is spiral wound. This introduces two other problems, co-current flow and axial flow. The former limits the reaction to a single stage while with the latter the interfacial layer is prone to stagnation.

Laminar flow is typically limited to microfluidic channels to achieve the benefit of high efficiency due to membraneless interfacial diffusion coupled with simplicity, as pumps are the only moving parts. Herein lie the fundamental limitations. Linear microfluidic channels are restricted to a few, generally two fluids that flow in co-current mode over short distances, commonly a few centimeters. As fluid drive is limited to the pump pressure that is maximal as wall resistance and pressure drop result in unwanted mixing and short-circuiting. Consequently overall surface area is sorely limited. This limitation necessitates a tradeoff between fuel wasting (in electrochemistry) and pump rates. Furthermore, co-current flow is limited to a single reactive stage.

In addition, size parameters and flow rate have to be recalculated and redesigned for fluids differing in viscosity, density, thixotropy, miscibility, phase and many other parameters. Hence such designs are neither flexible nor dynamic; they present single application solutions. A variety of referential numbers have been developed to describe the fluid performance—Reynolds number, Dean number, Peclet number, among others—to provide a guide for the laminar vs. chaotic flow regimes. The Peclet number is a preferred guide to limits of performance.

Despite small scale efficiency scale-up presents significant difficulties for these microfluidic designs. “Scale-up” of such designs is actually numbering up as many small units operate in parallel. This then introduces issues requiring careful control of fluid flows as small variances effectively broaden the performance peak.

A spiral channel can be used in place of a liner or serpentine design to benefit from the inherent formation of vortical flow. However, pump driven spiral flow using multiple fluids in a single channel leads to plug flow and only a single reactive stage can be realized as evidenced by the work of Hornung and Mackley [2009]. They show that pump pressure alone, even in a spiral tube is insufficient to maintain discrete laminae over any long distance.

Process intensification via centrifugal rotation was developed to improve contact and reaction rate while reducing apparatus size. The two most common methods are the rotating packed bed and the spinning disk or cone reactor. Forced mixing in rotating packed beds or on a spinning disk or conoid surface introduces new problems. The rotating packed bed is an enhanced closed stirred tank reactor that achieves micromixing by virtue of additional solid surfaces. The rotating disk or conoid surface creates surface waves, entrains lighter fluids and necessarily thins as fluid moves along the radius undergoing quadratic volume thinning. The essential problems are increased energy operating cost, phase separation, pressure drops, drag, flooding and dead zones. They also sacrifice laminar flow. As a general rule for both designs and many others like it each ten-fold efficiency increase is realized at a ten-fold energy operating cost; no differential benefit is achieved.

Despite their advantages over closed stirred tank reactors (CSTR) all of these design approaches have severe limitations and fail to realize long, large scale, membraneless thin film interfacial reaction surfaces. Further, they lack a high degree of flexibility. Zambri, a student of MacInnes, in his thesis [2014] detailed the limitations of current laminar flow and process intensification efforts. He then went on to describe a different process intensification design, a centrifugally rotating spiral. All of the Zambri discussion is incorporated by reference.

In sum, some of the desired goals are: membraneless flow, continued energy input to maintain flow pressure, enhancement of vortical flow, and thin film diffusional interfacial transfer. A centrifugally rotating spiral channel is the next step towards realizing these goals.

Three groups have addressed the design, operations, performance and advantages of spiral channels for controlled physicochemical process—Y. Ito at the NIH, P. C. H. Li at Simon Fraser University, and J. M. MacInnes at the University of Sheffield.

As long ago as 1980 Ito initiated the idea of a centrifugally rotating spiral for physical separations, directed at proteins and cell sedimentation [U.S. Pat. No. 4,182,678; U.S. Pat. No. 4,321,138]. His work included a gas-liquid nitrogen foam extraction of bacitracin [Oka et al 1989] and radial multilayer chromatography to sediment blood cells [Ito U.S. Pat. No. 4,425,112]. Li and colleagues designed rotating spiral CDs for DNA diagnostic purposes [U.S. Pat. No. 8,343,788 B2; Yu et al, 2013] Their device uses only micro quantities, the object being clinical diagnosis rather than synthesis or separation or any form of volume production.

Ito and MacInnes each showed that Dean vortices and Coriolis forces form as a consequence of spiral rotation with the exact structures a function of spiral radius, rotational rate and fluid viscosity. The MacInnes group created centrifugal arithmetic spirals to demonstrate distillation and absorption by use of immiscible fluids, e.g., gas, aqueous, organic or ionic liquid pairs; in their patent they claim a multiplicity of spiral designs [Allen et al EP1703970 A1]. MacInnes et al have focused on theoretical considerations of the dimensions of the spiral channels, their frequency, the rotational characteristics, etc. These studies demonstrated the validity of the theoretical model.

All three groups created mathematical models of a centrifugally rotating spiral channel array (i.e., spiral and body as a single unit) with special reference to constant flow velocity [Peng and Li 2008; Yang et al 2009; MacInnes et al 2010; MacInnes et al 2012].

In sum, the work of Ito, Li and MacInnes and their colleagues reveal the centrifugally rotating spiral to be the most efficient and versatile concept for multi-phase processing eclipsing earlier microfluidic and process intensification methods. The work of these three groups noted above is included in full by reference with emphasis on the underlying mathematics and modeling.

Despite the great advances noted this body of work has some significant limitations and misunderstandings. The current invention expands on this work and overcomes such limitations. For example, the prior art regarding centrifugal rotation of spiral arrays is limited to channels as spiral tubing or as embossments in or on a platen where the platen is integral to the body and all must rotate as one. Nor do they deal with spirals that do not rotate. There is no consideration for rotating fluids. Another example is that process limitation is restricted to sedimentation, absorption, chromatography and distillation; the last required in channel condensation to collect the fluid in liquid form, as no method was available to allow collection of miscible phases. Another limitation is their stated need to use immiscible fluids only. Yet another limitation is the absence of serial or sequential reaction operations or the integration of successive rotors for discrete chemical reaction processes or the opportunistic use of membranes when needed. Yet another limitation is the absence of exogeneous energy sources, the ability to capture energy from the spiral system, e.g., electrical or thermal, the absence of dynamic performance sampling or of feedback control for optimization. In addition, none of the existing designs lend themselves to significant scale-up, a critical feature if this technology is going to move from the laboratory and specialty niches to a larger application space. All of these limitations and many others are overcome by the present invention.

### SUMMARY OF THE INVENTION

The objectives of this invention are to provide apparatus, designs and processes that will enhance a wide variety of physicochemical separations and reactions by overcoming current inefficiencies in mass and heat transfer and by introducing flexibilities to optimize reaction processes. This will be done by means of fluid flow in an arithmetic spiral to result in vortical flow of one or more lamellae separated by differences in density independent of but aided by miscibility, viscosity, temperature and other critical physicochemical properties. This will be result in efficient, stable, scalable, thin-film reactors supplemented by appropriate catalysts, and exogenous energy supplies under design modes that allow operation in series or parallel. The present invention describes a multiplicity of spiral arrays, stationary or rotating, integral with the mounting body or independent of it, processes for managing the interaction of fluids and solids in such arrays, as well as their selective activation of the enclosed fluids, the requisite apparati to deliver and remove fluids from the arrays and the ability to sense and control said processes.

Fluids flowing in centrifugally rotating arrays layer into discrete radial lamellae based on density, independent of temperature or miscibility where each lamella exhibits Dean vortices. These vortices exhibit a screw-like procession along the channel axis that refreshes the interfacial surface while moving fluid from the center of a vortex to its perimeter. Under low Reynolds number conditions laminar flow is maintained. Such spiral arrays are stable under membraneless conditions. A device effecting this process is referred to as Vortical Thin Film Reactor (VFTR). These channels can include membranes located along the flow axis to effect multiple contacting spiral chambers if and as beneficial.

Similar vortical flow can be achieved with a single fluid in each discrete channels under both rotating and non-rotating conditions. Here membrane separators are placed between spiral channels. These designs can be stacked or formed from a spiral wound tubular or flat membrane array, the latter akin to a spiral-wound filter apparatus. Further, the spiral can be arranged in three dimensions or can recur in one or more planes.

Centrifugally rotating spiral thin film systems provide far more degrees of freedom and allow more complexity, sophistication of design and dynamic control than do non-rotating designs. Incorporation of a heavy phase pump within the rotating perimeter allows for pressure control where the heavy phase vapor pressure would not allow control otherwise. Benefits include countercurrent flow, a multiplicity of independent flows and extremely long stable channels. Three factors regulate the flow patterns for each fluid, i.e., the flow rate ratio of the respective phases and the relative thickness of the phase layers. These factors are: spiral radius, rotation rate and pressure gradient along the channel. Appropriate control of these parameters enables optimum mass and heat transfer. They also allow for adaptation for phase and solute systems of differing fluid viscosities, densities, and solute diffusivities as well as the respective interface equilibria. The apparatus design and performance can be optimized by design or by feedback in terms of mass and heat transfer for given fluids, pressures, flows, materials and geometries. Feedback can be managed automatically by means of sensors organized in closed-loop circuits based on input-output performance vs. modeled or target values.

We disclose a wealth of novel applications and constructs for chemical process and for the reaction apparatus to enable the utility of rotating spiral designs. We examine single fluids, mobile and fixed solids as might be suspended at the interface between adjacent fluids especially were the solids, including beads and particles that can act as or provide a surface for catalysis or for electrodes or for electron transfer and where the buoyancy of the beads or particles is under discrete control. These arrays can also be used to enable electrochemistry or to support flow batteries, flow supercapacitors, fuel cells or flow deionization or desalination, to provide only a few examples of the breath of this invention.

It is further possible to direct specific energy to a defined location of a given lamella to further effect a desired reaction. Similarly, energy may be removed as heat or electrons, for example. Fluids may be removed and replaced dynamically. We disclose the role of static and dynamic membranes, for example ion exchange membranes between adjacent fluids as an alternative to density profiles to maintain separation of miscible fluids. We have demonstrated these and more as vehicles to make mass and heat transfer efficiency available to a wide breath of materials and reactions and to make such apparatus eminently scalable.

The initial rotating spiral requires that the spiral and its attendant body are unitary and rotate together. It is also possible to construct a design wherein the spiral rotates while the body housing remains stationary. This reduces the number of rotating seals needed and provides an alternative to rotating disc or cone designs. It is further possible to rely on designs without rotation of the body.

Each of these embodiments and their variants enable a wealth of diverse reactions under flexible yet readily controlled conditions. The applications and apparatus include concepts not described but obvious to those skilled in the art and are included without exception.

Applicable fluids include but are not limited to gases, liquids—aqueous, organic, ionic liquid, sol, gel, suspensions, and eutectic materials.

Solids: Non-flowing surfaces, porous or solid, such as electrodes, can be used with each the membraneless or membrane-separated embodiments.

Reaction processes may include absorption, adsorption, desorption, desalination, distillation, sedimentation, group transfer and electrochemistry, or any combination thereof among others known in the art.

Communication: Electrical, photic, ultrasonic, radiological, microwave and other means of energy transfer or communication, that influence or sense reaction performance can be provided to such channels and transferred to the rotating element via conducting fluids, metallic wires or filaments, and fiber optic filaments among others. Similarly, energy may be derived from the reactions ongoing in the reactor.

Fluid communication: The rotating element may communicate with the stationary surround by means of a seal-free J-loop with fluidized conductors or via rotating or non-rotating seals and slip rings.

Sensors/Feedback: Sensors can be distributed in the apparatus or made available via viewing ports or transparent ends to allow detection of process status and to enable control of flow rates and rotational rates to optimize performance.

These properties can be used to advantage in numerous different constructs. For example, a multiplicity of rotors each supporting a multiplicity of channels wherein each channel supports a multiplicity of fluid flows and fluids interact via adjacent radially organized interfaces. Second, membrane separated rotors. A multiplicity of rotors where each supports a multiplicity of single channels wherein each channel supports a single fluid flow. A participatory membrane can be used to separate flows in adjacent rotors. Fluids can interact radially across the barrier material.

### BRIEF DESCRIPTION OF THE DRAWINGS

FIG. 1 illustrates how Dean vortices and Coriolis forces develop in a rotating spiral channel.

FIG. 2 illustrates Dean vortices enhancing diffussion within the channels above and below a membrane while under centrifugal force.

FIG. 3 illustrates a multiplicity of single channel rotor platens splayed along a vertically rotating axis.

FIG. 4 illustrates fluid flow options in spiral channels. In a first inset two fluids flow countercurrent under centrifugal and pump drive. A second inset illustrates two countercurrent flowing fluids with a buoyant bead self-aligning at the interface.

FIG. 5 illustrates two fluids flowing as discrete lamellae in a single channel whose walls are covered in electrodes that can be used to deliver or receive electrical energy or can provide a series of sensors for control processes.

FIG. 6 illustrates a single spiral channel in a platen where the channel has multiple discrete entry and exit points to enable the serial addition or removal of substrate or product from the rotor to enable serial reactions.

FIG. 7 illustrates a sensor system in a heavy phase exit port to detect the interface between light and heavy phases in the port, with special reference to gas-liquid absorption-desorption or distillation.

FIG. 8 illustrates a pump at the perimeter of a spiral to collect the heavy phase, with special reference to gas-liquid desorption or to distillation to overcome lack of sufficient pressure to deliver the fluid to the center for recovery.

FIG. 9 is an exploded diagram of two large membranes that are wound in a spiral with a heavy fluid feed and return and a comparable feed and return for a light fluid. Glue lines on the membranes divide it into channels and set the channel thickness.

FIG. 10 illustrates a rotating spiral in a stationary housing. The spiral is divided into multiple platens each interfacing a heavy and a light fluid.

FIG. 11 illustrates a multi-layered spiral, here two spirals separated by a single membrane to allow fluids of equal density that may or may not contain particulates to occupy adjacent spirals. Electrodes are available as needed oriented on the rotational axis to use this as a flow supercapacitor.

FIG. 12 illustrates a multi-layered spiral; here three spirals separated each by a single membrane to allow different fluids in each compartment and where the separation membrane play an active part in the reaction or separation. Electrodes are available as needed oriented on the rotational axis to be used in desalination.

### DETAILED DESCRIPTION OF THE INVENTION

Objectives

It is an object of the invention to generate stable, self-assembling, self-aligning, extended interfacial surfaces between one or more adjacent flowing fluids or a flowing fluid and a stable surface to enable diffusive physicochemical reactions. It is a further object to effect control over the interfacial contact, and of the rate and magnitude of diffusive contacting at adjacent interfaces, at least one of which is flowing, to maximize mass and heat transfer, to optimize reaction performance and to prevent unwanted mixing. It is a further object to control availability of materials that constitute thin film lamellar flow. Yet another object is to facilitate axial flow to minimize the length of the interfacial contact for the diffusional interchange to allow the reaction under consideration to come to completion or the process to be maximized. A further object of the invention is to allow serial and parallel reactions between adjacent lamellae such that the product of a first reaction may, for example, become a reactant in a subsequent reaction and that such processes can occur in a single apparatus. It is a further objective to support a multiplicity of reactions as a means of scaling the process. It is yet a further objective to deliver to or remove from the spiral lamellae any form of energy for useful, sensing or control purposes at any given locale along the spiral length. It is a further goal to enable rapid and protracted self-stabilization coupled with rapid replacement of lamellar contents both from the interior of a flow and from the exterior to achieve the best possible performance. It is yet a further objective to control the pressures within the apparatus even under conditions where the system pressure approaches or falls below the vapor pressure of liquids in a channel, especially at the outer edge of the rotor to allow conjoint use of gas and liquid in a single channel.

To these ends we describe a multiplicity of spiral array embodiments, twenty-five in all. They divide as 1) an integrated rotating spiral and platen in disc form and in spiral wound membrane form, 2) a rotating spiral in a stationary housing, and 3) a non-rotating spiral. Each of these designs can be membraneless, i.e., fluids that flow in adjacent laminae remain distinct and separated for the entirety of the channel length. Such behavior relies on flow promotion by centrifugal or pump driven flows operating on fluids that differ in density and as may be aided by differences in miscibility, viscosity, concentration or temperature. Alternatively, in each of these designs the adjacent spiral channel arrays can be separated by means of a membrane where the membrane has some functional contribution to the separation or reaction or other physicochemical process. In these cases as well driven fluid flow is beneficial and preferred. Membraneless embodiments are preferred but practicality demands alternatives that enjoy the other features described in this enabling disclosure. For each of these embodiments fluid flow can be driven, alone or in combination, by centrifugal rotation or pumped flow, the last primarily in membrane separated designs each channel containing only a single fluid. The maximal channel length before mixing occurs is shortest for pumped flow and longest for centrifugal flow, in the latter case it is easily in excess of 2 m. Pumped flow is limited to co-current mode while electromagnetic or centrifugal flow can be co- or countercurrent. Centrifugal flow can also accommodate stably the largest number of adjacent flows. Conversely, centrifugal flow demands the most complicated fluid delivery mechanism while the stationary channel is the simplest. The number of degrees of freedom is greatest with the centrifugal flow and least with the stationary channel. Thus, it is possible to select preferred embodiments among these for any given reaction or process at any given scale and level of sophistication for any given levelized cost.

Fluid Flow in Spiral Channels

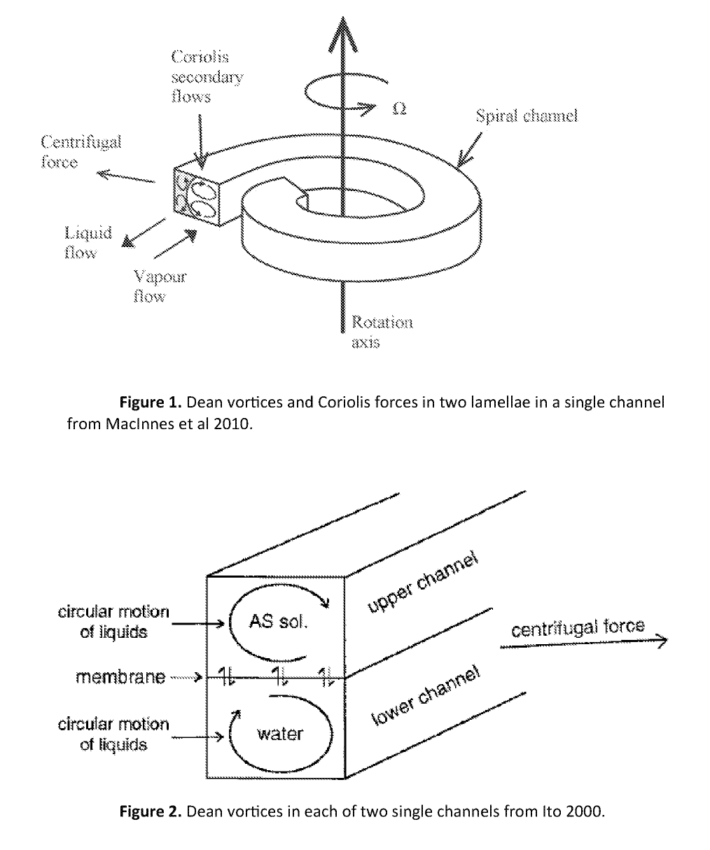

Fluid flow in small spiral channels is characterized by the operation of vortical flow, Dean vortices and Coriolis forces result in laminar thin film flow that enables diffusional transfer between adjacent interfaces, flowing or stationary; a preferred means to maximize mass and heat transfer and thereby optimize reaction processes. These are illustrated in FIGS. 1 and 2. Under these circumstances the bulk fluid, however modest, distributes to refresh the interfacial chemistry. Centrifugal rotation of an arithmetic spiral channel will produce these flows and thereby stabilize multiple fluids in a given channel. MacInnes, Ortiz-Osorio, Jordan, Priestman and Allen [2010] illustrated these processes for membraneless flow in a single channel (FIG. 1) while Ito [2000] illustrated the same for channels with membrane separation (FIG. 2). Our work has evidenced, for the first time, that stable flows in a single membraneless channel extend over distances>2 m, continue for many days in the presence of the centrifugal force and are achievable using miscible fluids that do not differ in viscosity, temperature or concentration. The prior art is limited to spiral flow of two immiscible fluids co- or countercurrent. We demonstrate stable co- and countercurrent flow using miscible fluids and we describe multi-fluid flows that include Newtonian and non-Newtonian fluids, thixotropic fluids, sols, gels, suspensions, etc. Stable flows will be maintained as long as the flow is maintained. Pumped fluid flow can achieve the same benefits albeit over far shorter distances.

Channel Characteristics

A channel is an enclosed or bounded space whose length is far greater than its width or height; length is defined as the axial dimension of the spiral, width as the dimension along the radial axis, perpendicular to the flow axis and height as the dimension perpendicular to both the radial axis and the flow axis, commonly parallel to the rotational or principal axis. (Note that MacInnes et al [2012] reverse width and height vis-à-vis the term usage here). Width may exceed height if membranes need a greater surface area. The channel can have a rectilinear or curvilinear cross-section or any combination thereof.

A channel can be made by the spiral wind of existing channels such as tubular stock. Alternatively it can be constructed into a platen where the platen is a discoid entity in two or three dimensions. It can also be the space enclosed by the spiral winding of flat membrane material where the channel height is bounded by means of glue lines or other such methods of creating boundaries. A platen can support one or more channels and channels may be interleaved. The channels may interconnect vertically or horizontally. Porous material may be inserted into the channel to provide contact sites, as an example to enhance gaseous reactions.

Channel width is the sum of the number and respective size of the several discrete fluid lamellae that flow in the channel. When at least one fluid is a gas it will expand to fill the available space. Lamellar thickness is a function of fluid viscosity, fluid flow rates, channel g-force and the radial g-force imposed on the fluid by virtue of its rotational flow.

Channel length is determined by the optimal interfacial reaction rate in terms of reaction time length, the number of theoretical stages desired, the relative flow directions (co-current or countercurrent) and the relative flow rates. This dimension is calculated for each fluid pair and for each reaction under given reaction conditions and must allow for any changes in the physicochemical properties of a fluid over the length of the reaction space. As an example, were a fluid to become more viscous during the reaction or it were to exhibit thixotropic properties.

A channel and a platen may be constructed or assembled by any known additive or subtractive method. Channels can be made as a single piece by 3-D printing or of discrete pieces that are each made by stamping, molding, etching, machining, rolling, extruding, printing or any other process known to the art. Further, rolling layered flat sheet materials can define channels allowing divisions to section the flows to the preferred channel dimensions. In the case of discrete pieces they are assembled to form an integral unit by means of gluing, thermal or pressure welding, assembly by fasteners of any kind or any other process known to the art. The assemblies can be fixed and permanent or they can be made to enable disassembly.

Rotational flow is given, in part, by fluid flow rate, which can be driven by pump pressure or centrifugal rotation each or in any combination. For example, under centrifugal drive, each channel is ˜100 μm to 10 mm in width, preferably <500-2000 μm. The channel height is limited for fluids flowing orthogonal to the Earth's gravity by the impact of gravitational force on the height of the fluid. Generally speaking the height is <50 mm, preferably <10 mm, more preferably 5 mm or even less.

Under centrifugal rotation of the integral platen and body design each flowing channel will require two connections—feed and product. These can be managed by use of two J-loop tubes or two rotating seals. The rotational axis of this design can exhibit any desired orientation—horizontal, vertical or at any angle; the difference in the Earth's gravitational field is of minimal consequence with horizontal rotation. In contrast, the rotating spiral in a fixed housing, an analog of the rotating disc/cone apparatus, requires only two sets of rotating seals and the desired number of slip-ring connections.

Contacts

The channels can include a variety of electrical, mechanical, optic, acoustic, vibrational, thermal or other means to deliver or remove energy from the fluid reactions or to sense and by appropriate circuitry control operation of the spiral apparatus. The presence of sensors, energy delivery or extraction processes e.g., electrical, optical, etc., will require the desired number of slip-ring connections for various connections. The apparati described can include a multitude of solid surfaces in uniform, segmented or prorated form along outer walls or set between various lamellae, as an example to provide reactive surfaces. One example of such art is the use of electrodes for the extraction of electrical energy or for the deliverance of electrical energy to one or more of the included fluids. The former enable fuel cell, flow battery, flow supercapacitor, or electrodeslination operations as but a few examples. Examples of sensors can include those for pressure, temperature, pH or any other parameter of interest or merit to monitor performance and to allow feedback control to optimize performance.

Spiral and Flow

An arithmetic spiral is preferred for channel pattern, more preferably a spiral pattern wherein the axial force is held constant along the length of the channel. Allen et al [2005] describe a wealth of other non-arithmetic spirals that may be applicable. Preferred layouts are: the Archimedean (arithmetic) spiral as described by Y. Ito [1982] where r=a+b*θ, the equiforce spiral as described by X. Y. Peng and P. C. H. Li [2008] where rₒ*cos αₒ=r*cos 60 , or the spiral described by J. M., MacInnes, M. J., Pitt, G. H. Priestman and R. W. K. Allen [2012] where r sin α=(h/2π)(1+t/h) or another way of calculating an arithmetic spiral. Other spirals that can be used include the involute of a circle where r=a/cos α, a Fermat spiral where r=θ̂0.5 or the dual Archimedean spiral in single or recurrent design. Spirals can be organized in two- or three-dimensional arrays and can be interleaved per X. Y. Peng and P. C. H. Li [2008]. Of these options the equiforce spiral is most preferred although the numeric differences in radial and axial forces are quite small between the three formulations particularly as the size of the radius increases.

For all of these spirals under centrifugal load the g-force against the wall needs to be significantly great, >5, to maintain the phases separate while maintaining a fairly constant g-force to move the heavy phase along the spiral. This condition is satisfied when α is small. Note that the greater the viscosity the easier it is for fluids to remain discrete.

Fluids flowing in an arithmetically wound spiral channel inherently segregate into discrete lamellae on the basis of density, miscibility, concentration, or thermal differences. Density is the dominant parameter with centrifugal rotation or spiral flow by other means. For readily miscible fluids such as aqueous salts under g-force of 50 or less we demonstrated a minimal acceptable difference between adjacent laminae of 0.023 g/ml. MacInnes et al [2010] report a minimal density difference of 0.013 g/ml for immiscible organics. Ito reported a minimal density difference of 0.001 g/ml and demonstrated performance of 0.005 g/ml for co-current cell sedimentation using a dense Percoll sol step gradient under a force of 170 g over a circular, non-spiral, length of 26 cm [Shiono Y, Ito Y 2003].

Centrifugal force from the rotation produces a strong segregation of the phases so they can flow in parallel layers with high interfacial areas. Layer thicknesses can be controlled to account for changes in viscosities, densities, diffusion coefficients and the required relative flow rates by altering the magnitude of the centrifugal force (via rotation rate) and the pressure gradient along the flow (via valve or pump settings).

Rotating flow designs have the advantages of dynamic control for optimization, extensive channel length, ability to organize mobile and stationary flows, ability to support multiple fluids under membraneless conditions in a single channel, the ability to support co-current or countercurrent flow of a multiplicity of fluids in a single channel and the ability to modify rotational rate and pump rate to control lamellar characteristics and reaction parameters. Channel width and wall contact angle can also be altered to advantage. Increasing RPM can reduce entrainment and thus increase capacity but at the cost of increased rotational energy and pressure drop.

Under and of the drive mechanisms with a corresponding non-mixing length fluid flow exhibits rapid start, stop and transition, i.e., assembly, disassembly or translation, due to the dynamic stability of the design. Each pair of adjacent phases, miscible or not, are forced to flow in parallel layers along the narrow spiral channel, side by side, co-current or countercurrent, the latter being preferred to achieve a greater number of theoretical stages for separation or reaction. This concept is applicable to larger channels, up to tens of millimeters in width and height and meters in length. By virtue of the screw-like Dean vortices and Coriolis forces material on the interior of a given segregated flow is rapidly raised to the surface; such segregated flows are referred to each as a lamella.

Selection of spiral parameters, rotation rate and pressure gradient along the channel, controls the flow rate ratio of the phases and the relative thickness of the phase layers. This allows ready adjustment to reach the optimum mass and heat transfer (limited strongly neither by one phase nor the other) and adaptation to phase and solute systems having widely differing fluid viscosities, densities, solute diffusivities and interface equilibria. With appropriate control over these parameters, a single device is capable of application to a wide range of separation requirements; such control is achieved automatically by means of sensors and feedback mechanisms whenever possible. In this invention we provide a multitude of reactor designs and operations to enable these objectives.

Membraneless laminar segregation is a preferred mode of operation and is achieved by opportunistic selection or formulation of fluids that differ in density as this allows segregation independent of the kinds and number of fluids used. The membraneless design has the distinct benefits of avoiding membrane resistance, membrane diffusional cost, low energy transfer, material, assembly and removal and replacement costs, fouling, and failing. Rejuvenation of static membranes requires in situ removal or replacement of active agent or deconstruction of the apparatus and replacement of the whole of the static material. The benefit of membraneless operation is especially evident as the reaction rate increases.

The thin films used in this invention can include interfacial dissolved catalysts for example provided as a separate fluid that will form a thin interfacial film. Suspensions can also be used, e.g., beads, particles and filaments whose buoyancy can be controlled to remain in a parent fluid or, preferably to self-align at a fluid interface. Such materials may be catalytic to the relevant reaction or may serve as a platform for the immobilization of a catalyst. In addition to delivering catalysts or being catalytic such particles may be conductive or reflective or exhibit any of a variety of atomic level properties of value in the ongoing processes. Examples of dynamic materials include but are not limited to copper particles, graphene tubes, plates, spheroids or rods, titanium dioxide particles or silica gel particles. Thus, a discrete thin film operates as a dynamic membrane and is distinguished from the static, mechanically stable membranes described below.

Inasmuch as catalysts, especially enzyme catalysts can foul or otherwise lose some or all of their catalytic capacity they would have to be replaced. This design is ideal for this purpose as given appropriate interrogation, e.g., a pH sensitive dye, the fluid, bead, particle, or filament can be removed from the flow as it exits the apparatus to be replaced by a new potent example of the same. This unique feature makes it fundamentally different from membrane constructs where failure of a channel or catalyst reduces the efficacy of the whole. This is readily accomplished using common cell sorting technology or modifications thereof.

Membranes

Membranes are a selective barrier that enables passage of some materials but not others. The term generally refers to a dimensionally stable material whose width and length greatly exceed their thickness, nominally a film. Membranes as used here are static structures that may be non-porous, microporous or nanoporous, for example made of plastics or metals. They may be size selective or permselective, i.e., semipermeable, for a given material such as a given gas, an anion or cation or an electron or proton. In the case of solid barriers they may separate energy transfer agents such as fluids whose temperatures differ, for example to provide heat transfer that may be delivered co-current or countercurrent to affect a given reaction between adjacent reaction fluids. They may allow the transfer of other radiation such as electromagnetic or ultrasonic to a given lamina in a given channel along its entirety or only a portion of its flow where such additional energy may facilitate a given reaction.

When a membrane separates two channels it is generally preferred if each spiral channel contained only one fluid with interfacial interaction at the membrane or electrode surface. The benefits of spiral fluid flow are shown in FIG. 2.

Fluids

The embodiments described below operate for all fluids and fluid mixtures including but not limited to gases, pure and mixed, and liquids the latter including aqueous, organic and ionic liquids, eutectic salts, liquid metals, polar and non-polar liquids, protic and aprotic liquids, sols, gels, suspensions and other ordinary fluids as well as supercritical fluids, indeed virtually any fluid. In the case of suspensions these may include beads or particles whose density matches that of the carrier fluid such that they are neutrally buoyant.

Reactions

A large number of different reactions and separations are supported given the apparatus and conditions described above and expanded below. Examples of such processes include absorption, adsorption, desorption, precipitation, sedimentation, evaporation, distillation, desalination, extraction, and a wide variety of redox and acid-base reactions, among others known to the art. A variety of redox reactions are supported by these embodiments, particularly under membraneless conditions. Examples include fuel cells, flow batteries and flow supercapacitors.

Further, as is evident from the figures such reactions can occur in the same apparatus sequentially in length or breadth introducing a level of complexity and sophistication not permitted heretofore in one continuous regime. Further, the ability to stack reactors that support discrete processes one can carry out a complete synthesis with a high degree of mass and heat transfer efficiency continuously, without manual intervention.

The advantages of the present invention include, without reservation, increase in reaction rate by design parameters of channel width, alpha angle and rotational rate while minimizing energy cost and channel density. This underlies improved reaction and separation rate and efficiency via mass and heat transfer between interfacial surfaces effecting scalable, stable, long length reaction interfaces that maintain thin-film (microfluidic-like) diffusional transfer advantages. These advantages hold for a very large variety of fluids to support a large variety of separations and reactions including a multitude of electrochemical processes. These apparati and processes result in minimizing reaction lengths, maximizing rates, maximizing specificity, and minimizing energy requirements.

The work of the research groups headed by Yoichiro Ito at the NIH, Paul C. H. Li at Simon Fraser University, and Jordan M. MacInnes at Sheffield University has each contributed to understanding the underlying mathematics involved. All of their work on this topic is fully included by reference.

### Embodiments

We describe three principal structural designs for spiral arrays each of which satisfies the requirements of being a vortical thin film reactor. They are: a) an integrated rotating platen/rotating body, b) a rotating platen/stationary body and c) stationary platen/stationary body with rotating fluids. These are presented in both membraneless and membrane containing formats. Examples of some of these are shown in the figures.

Some or all of these embodiments can be realized in membraneless or membrane containing designs. They can also be realized with one or more fluids flowing in each discrete channel, thus the reactive surfaces can be located on the radial or principal axis where the principal axis is generally taken to be rotatory. Membranes, when present, are used to carry out distinct operational functions, e.g., ion selectivity, permselectvity, or size selectivity, among others known to the art. It is possible in many cases to effect the same selectivity under membrane or membraneless conditions thus the design implemented is held on practical grounds, i.e., operational and economic optimization preferably based on the concept of levelized costs.

The presence of Dean vortices and Coriolis forces in fluids flowing in a spiral channel under centrifugal load is illustrated in FIG. 1 [MacInnes et al, 2010] for two fluids in a single channel and in FIG. 2 for one fluid in each channel here the channels being separated by a membrane [Ito, 2000].

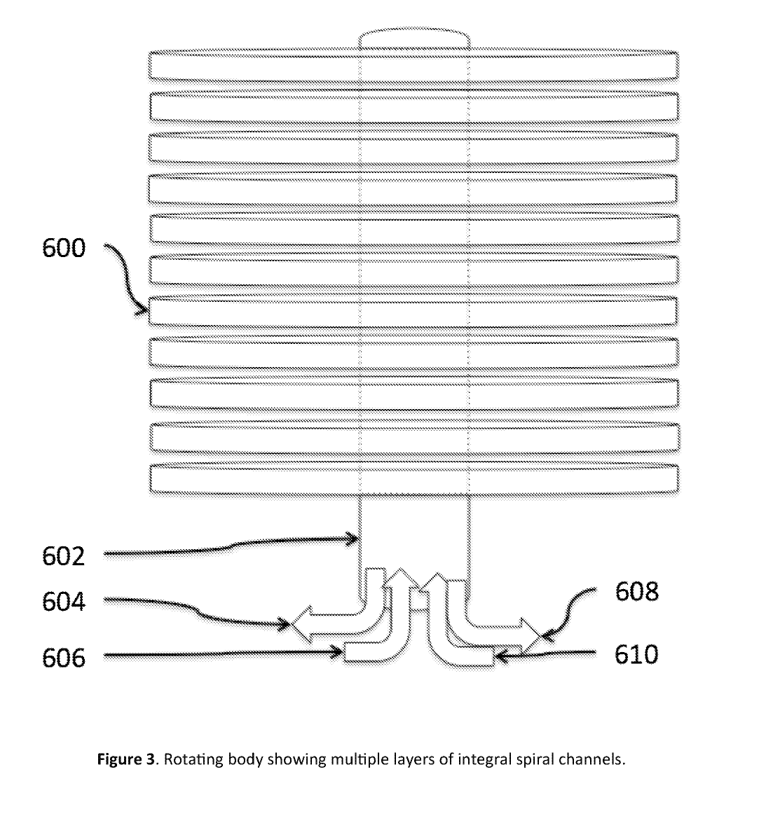

In the first set of membraneless embodiments the spiral channels are integral with the body of a centrifugally rotating apparatus where the body rotates on a central axis and where each channel contains one or more independent fluid flows. FIG. 3 illustrates a rotating unitary body consisting of a multitude of platens 600 integratively driven by a central axis 602. In a simple case two fluids react at an interface. The light phase enters at 606 and leaves at 604 while the heavy phase enters at 610 and exits at 608.

The rotational axis of the integrated body/spiral is independent of the gravitational field Earth and, indeed is independent of any gravitational field. Earth gravitational forces will act to limit the height of a vertically oriented array, particularly under lower imposed g-forces. The differences in gravitational fields imposed on a horizontally rotating apparatus, plus or minus 1-g are minimal in comparison to the imposed g-forces that are often in excess of 10-g and can exceed 50-g.

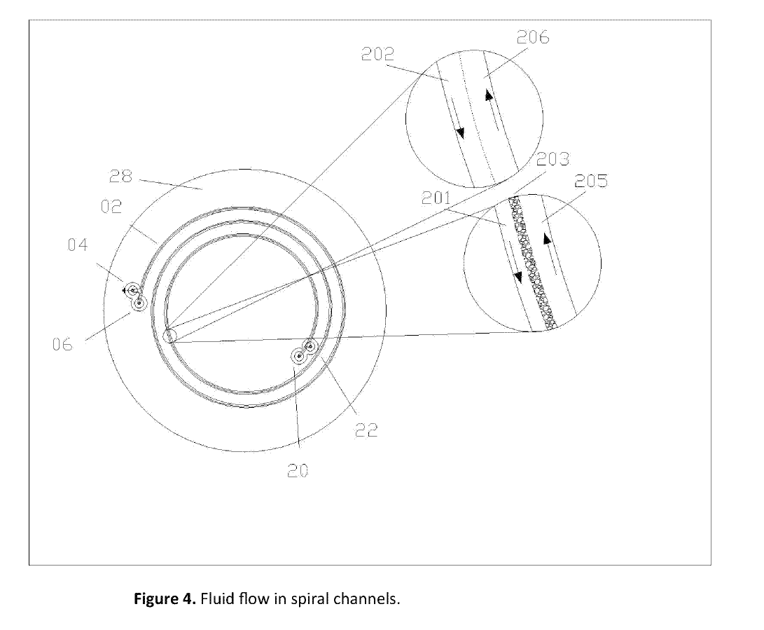

FIG. 4 illustrates a rotor platen 28 containing a spiral 02. The spiral contains an inlet for the heavy phase at 22 and outlet for this phase at 04. The light phase enters at 06 and exits at 20. A first inset illustrates two discrete flows 202, the heavy phase and 206 the light phase that flow countercurrent to one another. A density difference of at least 0.02 g/ml guarantees independence without regard to miscibility or phase composition. A second inset illustrates formation of a dynamic membrane 203 here in the form of self-assembling, self-aligning beads or particles whose density is selected or manipulated such that it is less dense than the heavy fluid self-locates centrifugally 201 but more dense than the light fluid that self-locates centripitally 205. Such materials can act catalytically or provide support for a catalyst. Thus, the beads, for example, form a dynamic interface which, given sufficient number, may form a relatively complete barrier. Any number of applicable flows can be inserted provided they satisfy at least the density requirement. Differences in miscibility, temperature, salinity, etc. can augment separation. The fluids can flow co-current or countercurrent with respect to one another—countercurrent with the lighter phase flowing outside-in and the heavier phase flowing inside-out with respect to the rotor. The heavier phase is preferred to flow head-to-tail with respect to the direction of rotation but optimal choices can be made

Energy Addition or Capture

Exogenous energy can be delivered to such rotating or non-rotating spiral arrays included among these being thermal energy, electromagnetic energy, vibrational energy and radiation where each can be delivered at a given locale or distributed along the length of the lamella and where such distribution can be uniform or distributed with a specific pattern; such pattern being uniform, punctate, structured or chaotic. Countercurrent thermal flow, commonly heat flow, can be applied to a fluid; which having absorbed a solute, for example a gas via physical or chemical absorption, for example carbon dioxide, that is now promoted to reconstitute and/or release said gas, i.e., to desorb the material. The same would apply to a packed solid such as an adsorbent, similarly to a flowable adsorbent, commonly particles, where by means of this invention continuous absorption/adsorption can take place in one zone and desorption in another as an alternative and improvement to the batch operation of swing-bed technologies thus eliminating one of the pair of swing-beds needed for pseudo-continuous operation. This process would apply to distillation and condensation as well. An alternative strategy would be to provide thermoelectric devices as opposed to thermal fluids. In the case of fluids given the thermal membrane separator any preferred thermally active fluid could be used having any properties desired for the process.

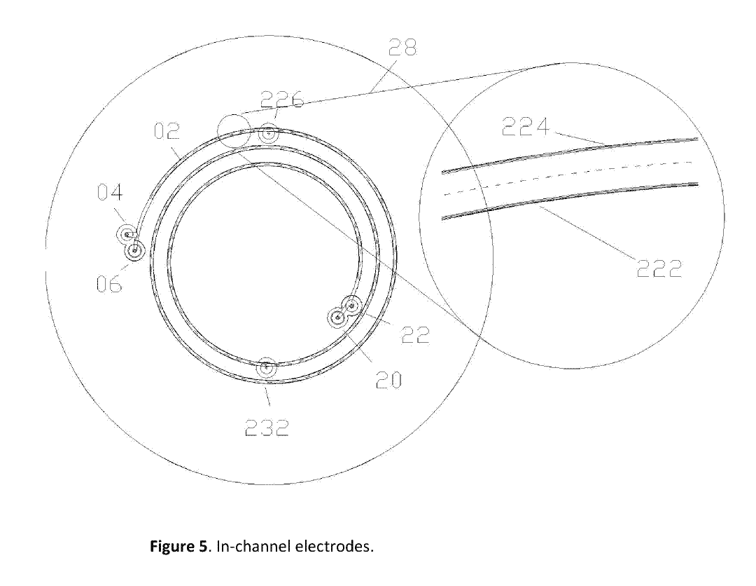

FIG. 5 illustrates a single channel 02 on platen 28 with a heavy phase entry at 22 and exit at 04. The light phase enters at 06 and exits at 20 for countercurrent flow. In co-current flow the inlets and outlets would be paired. The inset illustrates wall-mounted electrodes 224 located centrifugally and 222 located centripetally. These electrodes may be segmented and internally wired if need be to accomplish needed electrical demands. External connection points are shown on the platen as 226 for the centripetal electrode 222 and 232 for the centrifugal electrode 224. This electrochemical apparatus can capture or deliver electrons to the fluids thus operating, for example as a flow battery, fuel cell or as an electrically driven reaction process. The electrical connectors 222 and 226 can be joined by a solid or stranded wire or by a liquid conductor such as gadolinium that on exiting the channel is contained in a flexible tube. These same types of fibers, filaments or wires can be used to extract energy produced in the apparatus, for example any electrochemical operation or any thermal generation.

The use of the term electrode is symbolic and can represent any fiber, filament, wire or other materials that can be used for any of three purposes—energy delivery, energy capture or sensor monitoring of the reaction or process chemistry. Electrical, optical, magnetic, acoustic or other sensing or communications apparatus may be included in the channel or adjacent to it either for detection, addition or extraction of energy. As an example, ultrasonic energy imparts a five-fold improvement in desorption of absorbed gases, here a very effective method for removing carbon dioxide. Monitoring operations through appropriate sensors and circuitry can effect dynamic control of the apparatus. Examples can include sensors for temperature, pH, light intensity or wavelength and so forth.

In situations in which electrodes are included in a single channel they may be installed into channels that are open during manufacture and closed in use or they may be printed, for example by means of multi-materials 3-D printing methods, or they may be deposited, for example by plating, or delivered to the surface by any method, additive or subtractive known in the art. In the situation in which the construct is made of spiral wound membranes the inner aspect of the wound membrane is made of a conductive material. Energy transfer can also come via fluidized particles provided their density properties allow them to orient centripetally and centrifugally.

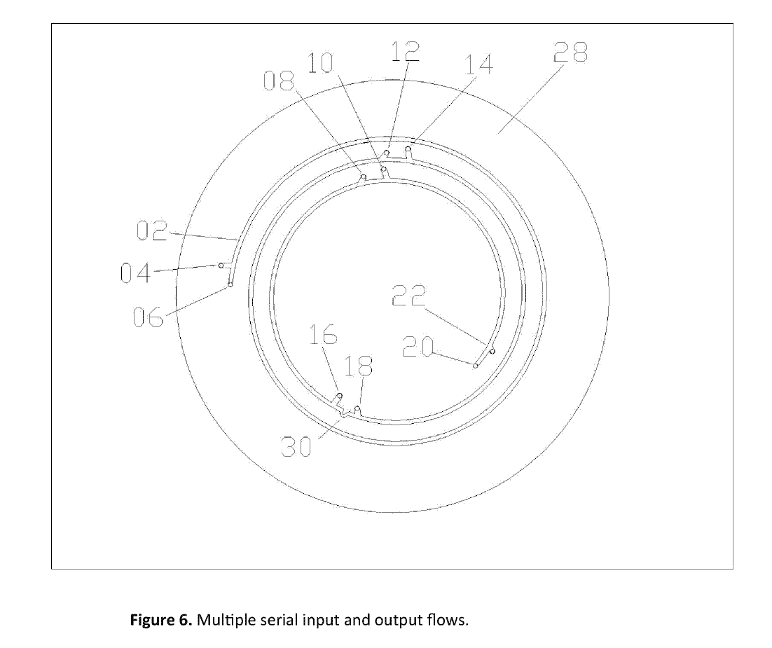

FIG. 6 illustrates a single channel in a rotor where the channel has multiple axially aligned inlets and outlets. Spiral 02 is present in rotor 28. The centripetal inlet and outlet ports are 06 and 20 and centrifugal ports 04 and 22. Under countercurrent flow conditions the light phase flows in the centripetal lamella while the heavy phase flows in the centrifugal lamella. This figure illustrates a multiplicity of entrance and exit ports to allow selective addition and removal of fluids from the heavy or light phases. Five port sets are shown that allow two light centripetal fluids to contact three discrete heavy, centrifugal fluids in support of serial reactions. For countercurrent flow the light phase preferably enters at the perimeter 06 and exits towards the center 18. A second light phase may then be introduced at 16 to exit at 20. The heavy phase enters at the center at 22 with an exit at 10. A second heavy phase enters at 08 to react with the same or a modified light fluid. It exits at 14 to be replaced by a third heavy phase at 12 that finally exits at 04.

The ports are so placed such that the light phase reacts with the third heavy phase over the length from 04 to 12. This second heavy fluid exiting at 14 can enter at 08 before being replaced by the first heavy fluid that enters at port 10. Similarly, the light phase flow can be segmented and serially replaced moving in a first case from port 06 to port 18 where it is removed while a new fluid is injected at port 16; it then proceeds to port 20.

In other examples all, a subset or a superset of secondary, tertiary, or quaternary ports can be provided per the examples here of ports 04, 06, 20, and 22. This design can be used for co-current or countercurrent flow. A jog in the channel 30 allows the heavy fluid to provide a seal between the light phase exiting at 18 and entering at 16. This can control for any pressure difference between exiting and entering fluids.

The entrances and exits shown in the prior figures are planar with the spiral but they can be orthogonal to access to a multiplicity of fluids in excess of the two illustrated here. Further, the spiral can be organized in three dimensions and other spirals interleaved.

Channel Operation

When a channel contains only incompressible fluids they may be pressurized into the channel from outside the rotating equipment. The actual pressure within the spiral is usually not significant for the reactions taking place in the spiral but can be relevant to structural requirements of the spiral. The distribution of the exiting incompressible fluids is determined by controlling the rate of fluid discharge from all the exiting fluids but one. That one will be the balance of the systems volume and will set the pressure basis for the spiral system. An alternate use of the uncontrolled stream is to adjust the flow out to achieve a design pressure and thus the system pressure. This is accomplished by restricting the flow to increase the system pressure or pumping out to decrease the system pressure. The pump out is limited to the vapor pressure of the fluids throughout the system.

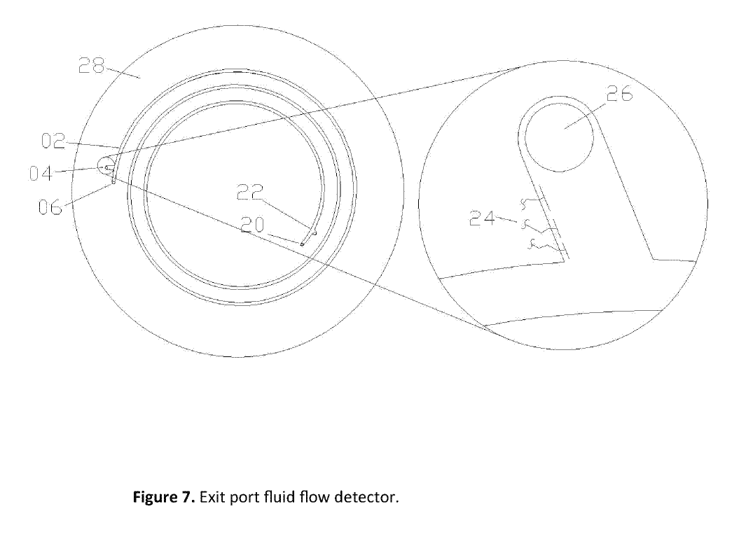

FIG. 7 illustrates a sensor system located in the exit port 04 of the heavy (liquid) phase of multi-fluid flow channel. The sensor detects a liquid exiting the light phase channel. Centripetal ports are located at 06 and 20 and centrifugal ports at 04 and 22 on spiral 02. The inset detail illustrates an exit channel in a gas-liquid absorption channel, or any channel where the heavy phase level in an exit port will be needed to control spiral operation. The blowup of port 04 illustrates flow detectors 24 for monitoring the exit flow at portal 26. The three detectors operate by virtue of a nulling circuit (not shown). A signal to the nulling circuit can trigger changes to the speed of the feed or exit pump to prevent unwanted mixing. The detectors can be electrical, optical or use any other acceptable and demonstrable method to effect this control and thus insure stable flow for protracted periods as might be influenced by changes in fluid viscosity, minor fluctuations in pump rate or rotational rate.

Pump

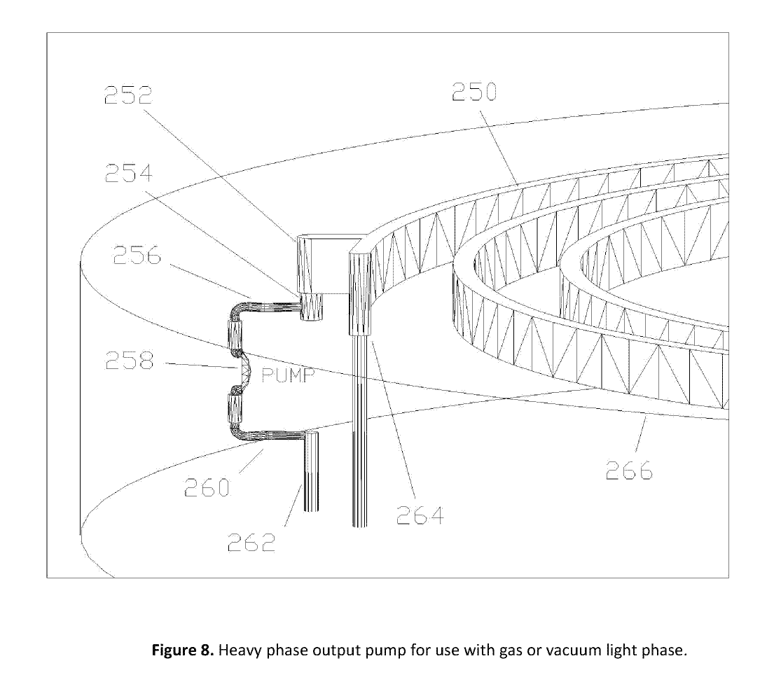

The fluid dynamics change when one of the exiting fluids is compressible. Under these conditions, as illustrated in FIG. 8, additional pressure must be delivered to the heavy phase at the perimeter to drive the fluid back to the center for removal or redirection. In this case the feed fluids are metered at the inlet and the exit liquid is pumped out using an external pump (FIG. 8) at a rate just sufficient to prevent the compressible phase from leaving with the liquid phase. The compressible phase is then pumped out at a rate consistent with controlling the operating (design) pressure of the spiral discharge. If the design pressure at the incompressible fluid exit approaches the vapor pressure of the incompressible fluid then the incompressible fluid could not be pressurized sufficiently to reach the center of the centrifuge; this would compromise the design spiral pressure. In this case the exit pressure of the incompressible fluid must be increased to enable it to be discharged through the center of the rotor. We have incorporated a pump in the rotor to achieve this end as shown in FIG. 8. With the pump we can operate the spiral at any design pressure by pumping the incompressible fluid out at a rate necessary to accomplish the design conditions. The liquid will then evaporate to the extent allowed by the design; it is then pressurized in the rotating body to the extent needed. The reason the liquid flows further toward the centrifuge periphery is to achieve the required Net Positive Suction Head (NPSH) when it enters the pump suction; it is then pumped to the rotor discharge, or to any other point on the rotor.

Under conditions of desorption as noted wherein the vapor pressure of the liquid may exceed the operating design condition. The apparatus illustrated in FIG. 7 can detect liquid level to dynamically control the liquid flow rate. Under these conditions, as illustrated in FIG. 8, additional pressure must be delivered to the heavy phase at the perimeter to drive the fluid back to the center for removal or redirection.

FIG. 8 shows an exit channel pump apparatus for channel 250 set in rotor 266. Centrifugal lamella 252 exits via tube 256 to pump 258 whose flow exits via tube 260 into tube 262. The centripetal flow may enter via port 264 and exit from the centripetal connection not shown. The rate that the vapor discharge pump operates is just sufficient to maintain the design pressure profile. This may be inferred by measuring the external to rotor discharge pressure or by internal pressure or temperature sensors.

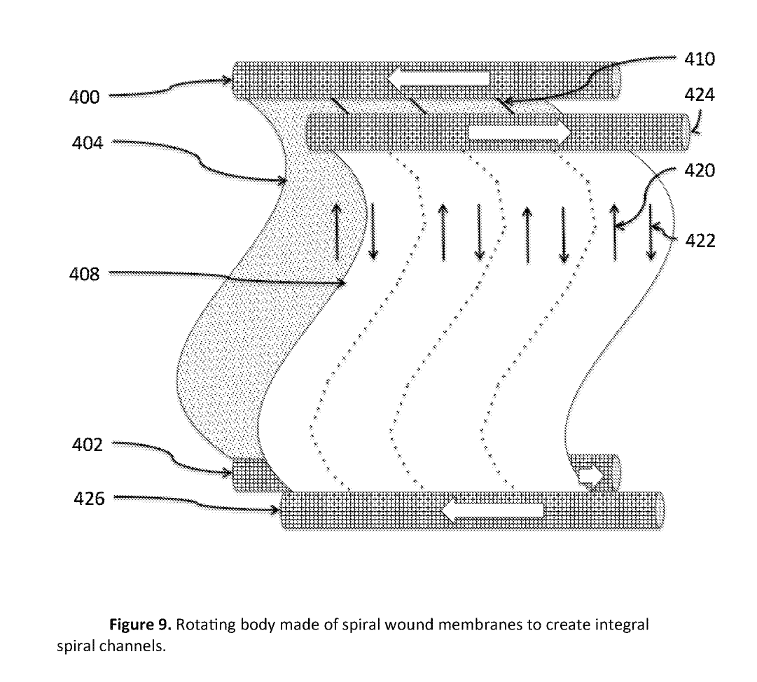

FIG. 9 illustrates a rotating unitary body assembled by means of wrapping rectilinear membranes into a spiral array. A first membrane 404 and a second membrane 408 define a space where the heavy fluid is delivered via supply tube 402 and the processed heavy fluid is recovered in. The light phase is delivered by tube 424 and recovered by tube 426. The heavy flow direction is shown by arrow 422 and the light flow by arrow 420. The fluid flow space is divided into segments by glue lines 410. The rotational axis is horizontal. This allows single construction on the order of 1-2 meters in length or more and 0.3-0.5 m in diameter or greater as may be desired and supported by the invention. This is an example of scale-up previously unable in membraneless designs

In each of the figures illustrated spiral channels can be independent or they can be linked via internal flow or plumbing, in so doing their dimensions can be changed to effect different flow rates.

Fluid Flow in a Spiral Array

In a second set of membraneless embodiments the platen containing the channels is free to rotate independent of body, which is fixed to a horizontal orientation. Any of the methods noted above or others known to the art can manufacture the spiral as well as the housing.

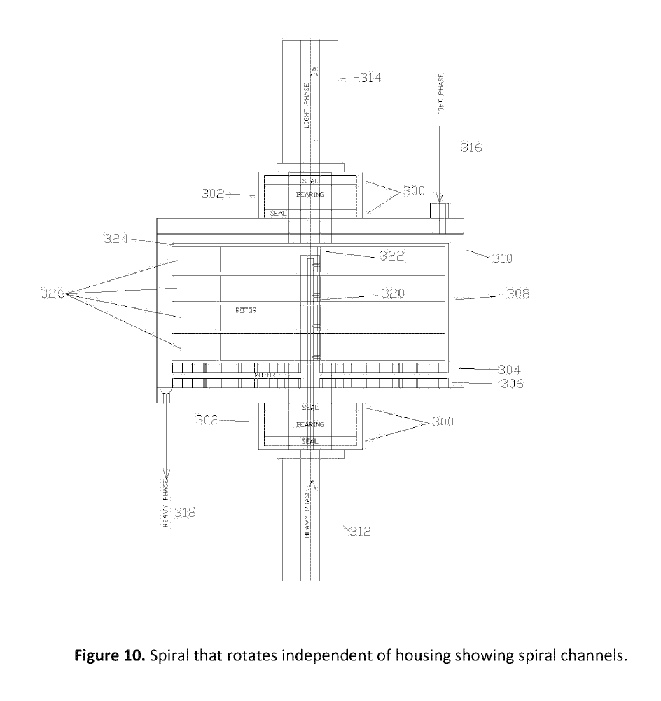

FIG. 10 illustrates a single rotating platen 324 in a fixed housing 310. The housing 310 has two sets of seals 300 designed to protect the bearings 302 that stabilize the rotor platen and keep the fluids entering and leaving the rotor separate from the fluids in the fixed housing. The heavy phase passes through the fixed supply pipe 312 that then distributes into the rotating spiral channels 326 by ports (holes) via distributor 320. The light phase exits via a space circumferential to tube 320 and the centripetal end of the channels 326 and flows up through the rotating section and into the fixed exit pipe 314 via entry ports 322. Exit of the heavy phase occurs from the housing body at port 318; the light phase enters of at 316. The light phase enters the periphery space 308 of rotor 324 and is pressurized into the spiral channels where it contacts with the heavy phase exiting the channels 326. The heavy phase collects against the fixed body 310 to fall by gravity to a grove in the base that conveys the heavy phase to the exit at 318. An external magnetic drive 306 rotates the platen that has an embedded magnet in its base 304. Four spiral channels are shown. Slip ring connectors (not shown) are available for sensing, signaling and delivery of specific energy where and when needed.

The platens and their attendant parts including but not limited to covers, guides, etc. stationary or dynamic, can be assembled by mechanical means, gluing, welding or any other method known to the art or may be assembled in whole or in part by dynamic means under forces such a rotational, magnetic, pressure, among others known to the art.

It is obvious to those skilled in the art that this design is a significant improvement on spiral disc or spiral cone designs in that it imposes exacting control on the flowing fluids and allows electromechanical connections (not shown) to enable the delivery or capture of energy or of informationally valuable signals to control performance of the device.

Membrane Reactors

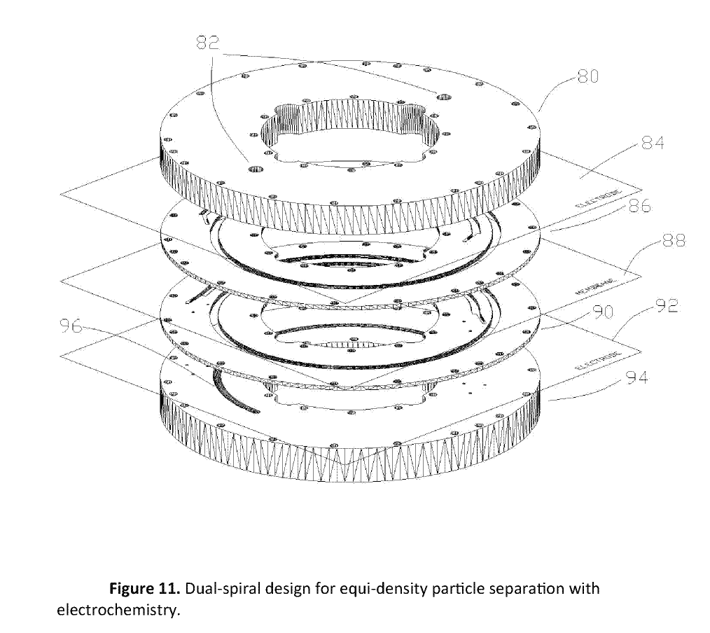

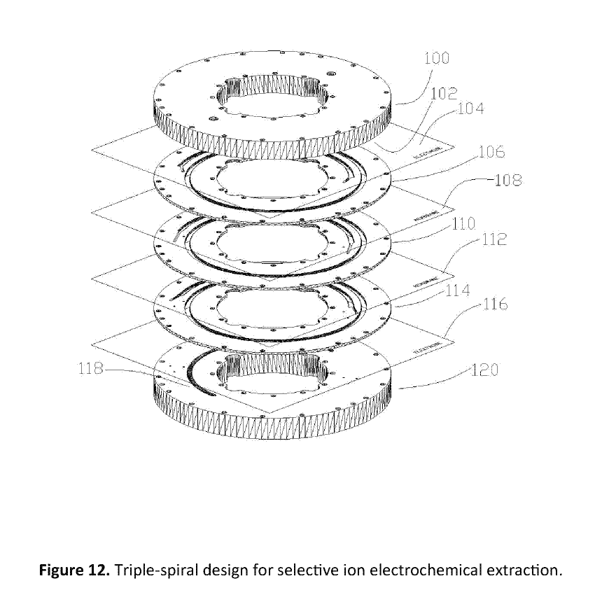

The preceding illustrations are examples of membraneless designs. Spiral channels can also be separated by means of membranes of various kinds as illustrated in FIG. 2 and described below. In these embodiments selective membranes separate one spiral from another where the membranes are selected to serve active functions in the reaction processes and where they are located perpendicular to the rotational axis (CF. FIGS. 11, 12); the apparatus may or may not rotate, preferably the fluid flows in a rotational manner. The spiral rotation induces Dean vortices that refresh the membrane interface improving mass transfer.

FIG. 11. In the dual channel membrane separated embodiment a barrier separates the flows moving in mirror image registered spiral channels. One such embodiment, for example, would involve graphite particles in both fluids; this design would be applicable to flow supercapacitors. In this case graphite particles flow in a support medium containing carbon black. Electrodes register the discharge electricity. These same electrodes are also used for recharge.

In a triple rotor embodiment two separation barriers keep separate the fluids in each of three adjacent spirals. FIG. 11 illustrates two spiral rotors, 86 and 90 each open on both lateral surfaces, with two end caps 80 and 94. A permeable or semi-permeable membrane 88 separates the rotors from one another, while the rotors are separated from the end caps by electrodes 84, 92. A conductive liquid or highly flexible multi-filament wire is used to contact the electrodes and allow for the transmission of electrical energy from the exterior when using a J-loop. Energy is transmitted to contact channel 96 then to the underside of end cap 80 (not shown). This power comes through connectors on 80 at point 82 and on the underside of 94 (not shown). A solid wire connected to a rotating contact can be used when using a rotating seal. Rotor 94 contains connections to pass fluids into and out of spirals 90 and 86. Fluid passage holes are in 94 and 90. The electrodes may be continuous or segmented and may be made of any preferred material with examples including but not limited to solids, porous foams or surface treated to increase the available contact area.

The triple rotor embodiment, illustrated in FIG. 12, could be applicable to electrochemical flow desalination. Here the center channel set contains the rich-feed while the adjacent channels hold the cations and anions respectively carried in a fluid or on ion exchange resin beads or on graphite beads and where the electrodes on either side draw the respective opposite charged species from the rich-feed to enable a lean-product. One example would desalinate ocean or brackish water for industrial, commercial or potable use.

The reactor in FIG. 12 illustrates three spirals 106, 110, 114 each open on both lateral surfaces, separated by ion exchange membranes 108, 112. The outer two rotors 106, 114 are separated from the end caps 100, 120 by electrodes 104, 116. Fluid wire channel seen as 118 and not seen on the underside of 100, connect via connectors, seen on the top of 100 and not seen on the bottom of 120 to flexible tubes to the outside when using J-loop connections where power can be supplied to the electrodes 104 and 116. A solid wire connected to a rotating contact can be used when using a rotating seal. Again connectors in 120 are not shown. These connectors also provide fluids to spirals 106, 110 and 114. The passage holes are visible in 120, 114 and 110. Through these connectors a salt feed could be fed to 110, electrical energy fed to the electrodes 116 and 104 and sweep fluids fed to 114 and 106. From this system a purified solution could be removed from 110 and anion and cation enriched fluids removed from 106 and 114.

### EXAMPLES

CO2 Capture Studies

a. Absorption

Carbon dioxide can interact with solutions by means of physical and chemical absorption or by adsorption in the case of suspensions. In physical absorption the CO2 molecules intersperse between the solvent molecules. In chemical absorption the CO2 reacts with a base, commonly an amine or hydroxyl groups, to form carbamates, carbonates or bicarbonates. A variety of enzymatic, organic and inorganic compounds can potentiate this reaction. Such facilitators can be delivered as a dense liquid, as catalyst particles or as catalyst (or biocatalyst) embed in or adsorbed onto a surface, as some examples.

We examined the rate of CO2 absorption into a potassium carbonate solution in the presence and absence of carbonic anhydrase when using a membraneless spiral centrifuge VTFR. The results were compared with those obtained using a microporous hollow fiber membrane design. Both operated under countercurrent flow conditions. The dimensions of the spiral channel were 3.9 mm W×2 mm H×704 mm L; the spiral was rotated at 750 RPM. The interfacial area of the fluid stream was calculated to be 0.001408 m². A 0.01M potassium carbonate fluid flowed from inside out at a rate of ˜2 ml/min (0.6 μM CO3=/s or 36 μM/min). CO₂-rich gases, 13.53% and 7.9% concentration, flowed countercurrent, outside in, at rates varying from 0.00033-0.00713 mol/h (5.5-118.8 μM/min).

The data shown in Table I reveal a dramatic improvement of more than one order of magnitude using the VTFR as compared to the hollow fiber design. Note that compared to other absorption techniques the hollow fiber is already very efficient. Thus, the VTFR is the most efficient design known to date. The mass transfer benefit, ˜80%, is seen by the decrease in the additional benefit provided by the enzyme catalyst carbonic anhydrase. Under these conditions the entire interfacial surface was utilized while the hollow fiber design provided only 40% open pore space significantly reducing contact area.

TABLE 1
CO2 Absorption Comparison - VTFR vs. Hollow Fiber

| | HOLLOW | | Apparatus |
|---|---|---|---|
| UNIT - 1e⁻⁹ mole/m² s Pa | FIBER | VTFR | Improvement |
| Without CA | 3.75 | 66.6 | 17.76 |
| With CA | 6.48 | 89.1 | 13.75 |
| Enzyme Improvement | 1.73 | 0.34 | |

In the embodiment described desorption was effected by means of a vacuum. In an alternate embodiment ultrasonic energy can be delivered to the CO2-rich fluid; it enhances desorption by 5-fold.

Proton Transfer Studies

Fuel cells and flow batteries commonly use aqueous fluids that are separated by a proton selective membrane. As described above, membranes pose several unwanted problems—cost, assembly, diffusional resistance, decreased effective contact interface, fouling and failing by shorting and by tearing. A principal reason for their use is to maintain the two fluids separate, under non-vortical flow conditions while allowing the selective transfer of protons. Laminar flow designs have clearly demonstrated the absence performance superior to any benefits provide by a proton selective membrane provided the fluid flows remain separate. As noted, co-current flow as is used in microfluidic devices limits efficiency to only one transfer stage whereas countercurrent flow enables multiple transfer stages.

In this work we demonstrate the efficacy of proton transfer, a proxy for electron transfer, over an extended length and thus surface area. This was accomplished by means of a membraneless single channel design; co-current and countercurrent flows were compared. We further demonstrate that miscible aqueous fluids are readily used in such a paradigm, exhibiting reliably controlled reaction mixing.

To this end we constructed a pair of miscible aqueous solutions, a strong acid and a strong base, that differed in specific gravity. A pH sensitive dye was added to the basic solution to provide indication of acidification, to provide color contrast for photographic purposes and to easily visualize the maintenance of discrete stable laminae. Photographic analysis demonstrated that density differences held the fluids apart for at least a length of almost one-half meter (the length of the channel) allowing only interfacial contact and exchange.

Flow rates varied from 1-4 ml/min into a channel 3 mm W×3 mm H×474 mm L. The rotation rate was either 550 or 910 RPM. Solution pairs consisted of 1M HCl vs. 1MKOH and 0.01MHCl vs. 0.01MKOH. The heavy flow thins as a consequence of the increased RPM comparing flows at 910 vs. 550 RPM. However this did not appear to result in a significant difference in proton transfer. The pH of the flowing solutions was measured accurately and continuously. Fluid flow variants included co-current or countercurrent, outside-in or inside-out.

Proton reduction varied significantly depending on flow rate and solution concentrations. As an example 1M solutions with a density difference of 0.031 g/ml, flowing co-currently at a rate of 1 ml/min exhibited a 29% proton reduction. In contrast, 97% proton reduction was observed using 0.01M solutions with a density difference of 0.034 g/ml, in co-current flow at 910 RPM and a pump rate of 1 ml/min. In contrast the proton reduction was 87% when using countercurrent flow with 0.01M solutions with a density difference of 0.034 g/ml, rotated at 910 RPM flowing at 1 ml/min.

These data demonstrate the ability to maintain two miscible aqueous fluids flowing in discrete laminae over a length of almost 0.5 m while exchanging protons in a controlled and highly efficient manner. This means this approach using the vertical thin film reactor avoided fuel wastage, a commonplace in linear microfluidic laminar flow reactors.

Electrochemical Flow Capacitor Using VTFR

Flowing activated charcoal-based electrochemical capacitors have performance characteristics similar to those of fixed supercapacitors with the advantage of a virtually unlimited fluid storage capacity. The design of the flow capacitor cell and the flow channel in particular, play a critical role in the performance of the electrochemical flow capacitor. Performance is governed by several factors including the energy window given by the nature of the electrolyte or the capacitance of the active material. Increasing the rate of redox reactions at the electrode-electrolyte interface will improve the capacitance of the material. It is here that the benefit of enhanced mass transfer is realized.

To that end we designed the spiral centrifugal rotor as shown in FIG. 11. It consisted of two rotors separated by a size-exclusion membrane to prevent the activated charcoal particles, ˜2 μm in diameter, from crossing from one spiral to another as they differed in specific gravity by ˜0.02 g/ml. The activated charcoal particles were suspended in a conductive fluid −0.139M p-phenylenediamine redox mediator in 2M KOH. The object was to use the phenylenediamine/p-phenylenediimine (PPD/PPI) redox couple for additional storage of energy. The solid-to-liquid ratio was 23 wt %; water was added at <5 wt % to enhance flow. The specific gravity was the same for of each of the two fluids where one would become the negative and the other positive. As shown in FIG. 11 flat sheet electrodes were placed at the uppermost and lowermost boundaries of the spiral channel set each just under the cap that carried the wire or fluid, e.g., galinstan, which contacted electrodes in each of the rotor spirals.

One advantage of this design is that multiple independent spiral channel sets can be linked to enable sequential charging and discharging to extract maximal benefit.

The apparatus was targeted to rotate at 910 RPM. The vortical flow of fluid in each rotor results in continuous refreshment of the membrane surface as well as the electrode surfaces. The electrodes that separate the rotors from the end caps serve to remove or add charge to the carbon particles so as to provide power or to recharged the flow capacitor.

The results of such an experiment would be expected to yield the following: energy density of 3.7 Wh/l coulombic efficiency of ˜98%, the capacitance was ˜150 F/g.

A redox-mediated slurry is a facile method for increasing the energy density of the flowable electrodes for the electrochemical flow capacitor. A pseudocapacitive slurry that utilizes a PPD/PPI redox-couple in 2 M KOH electrolyte would demonstrate high capacitance for the electrochemical flow capacitor. The alkaline redox-mediated slurry should increase in capacitance by about 86% as compared alkaline solutions without redox mediators, and a 130% increase compared to previously reported neutral slurries. The rate handling performance of the redox-mediated slurry capacitance decay should improve by about 7% between rates of 2-100 mV/s. The redox-mediated slurry should decrease the ohmic resistance, with increasing concentrations of PPD; this can be described by the increase in the ionic diffusion coefficient. A concentration of 0.139 M of PPD in 2 M KOH is expected to yield the highest capacitances in over a wide range of charging rates. The high performance would then be attributed to the addition of quick redox reactions at the electrolyte/electrode interface as PPD undergoes a two-proton/two-electron reduction and oxidation during cycling. The rapid surface refreshment in the VTFR is an important factor in maintaining and enhancing such performance.

The design and performance flexibility of the VTFR designs provides huge benefits over standard linear flow. This is important because the overall length of the channel determines the charging rate and the residence time of the slurry within the cell. Further, when using metal oxides it is known that the inner region is characterized by slower diffusion dynamics, due to extended diffusion lengths in comparison to the surface accessibility, hence the benefit of the VTFR flow.

Membraneless VTFR Electrochemical Flow Capacitor

Membraneless performance could be achieved using a design as in FIG. 5 where the specific gravity of the support fluids and beads has be structured such that they differ by more than 2 parts per hundred. Then charge separation will occur, as was demonstrated for the pH study. This embodiment exhibits the same characteristics, as does the “Electrochemical Flow Capacitor Using VTFR” discussed above. The principal difference is the expected improved performance due to the absence of a membrane. The capacitance is expected to increase to >200 F/g with yet further increase expected in the future.

Electrocapacitive Desalination with Membrane VTFR

An apparatus was constructed as in FIG. 12 with three spirals separated by two ion exchange membranes; the distal end of each of the outermost spirals contacts a membrane electrode. A saline feed water is used as an example of a deionization process applicable to a wide variety of chemistries. In this design the ion-rich fluid, here salt water, flows in the center spiral. A DC charge is placed on the electrodes such that the cations pass across a cation exchange membrane towards the cathode and in so doing adsorb to the flowing activated carbon. At the opposite side the anions under influence of the positive charge flow across an anion exchange membrane to adsorb onto a separate flowing activated carbon layer. The centrifugal rotation forms Dean vortices and Coriolis forces that drive the pumped fluids along their respective spirals. The adsorbent fluids flow countercurrent to the rich salt water. The vortical flow underlies thin film contact between the conducting adsorbent fluids and their respective electrodes and ion exchange membranes. The thin films allow maximal uptake and the vortical flow refreshes the interfacial contact. Overall the process allows rapid, continuous separation without need for periodic charge reversal to reactivate the electrodes.

The experiment would be run as follows: A sodium chloride solution (32.1 g/l) would flow through the center spiral rotor at a rate of 1 ml/min. A 5 wt % suspension of activated carbon particles would flow through the outer spiral rotors countercurrent to the saline flow while the electrodes imposed a charge of 1.2V. Salt removal would exceed 95% with an efficiency of at least 12.4% at this high salt concentration and a minimal removal rate of 0.4 mg/min*cm̂2. This type of system would perform far better than KOH electrolytes or neutral electrolyte slurries. The total energy for making freshwater from brackish water is expected to be <0.6 kWh/m̂3, far lower than any alternate process and salt removal efficiency is more than 10-times greater than in typical flow-by cells.

While the foregoing written description of the invention enables one of ordinary skill to make and use what is considered presently to be the best mode thereof, those of ordinary skill will understand and appreciate the existence of variations, combinations, and equivalents of the specific embodiment, method, and examples herein. The invention should therefore not be limited by the above described embodiment, method, and examples, but by all embodiments and methods within the scope and spirit of the invention as claimed.

1. We claim: Vortical flow of at least one fluid flowing in an arithmetically coiled spiral channel where such flow is non-chaotic and is driven by exogenous energy such that the fluid(s) exhibit least one stable, vortical, thin film whose length is greater than 10 mm and where laminar interfacial flow ensues between adjacent surfaces, flowing or static.

2. Vortical flow as in claim 1, driven by means of an exogenous energy drive selected from the group consisting of: a) pump pressure at the channel inlet or inlet and outlet, b) vacuum pressure, c) centrifugal rotation of the channel, d) electromagnetic forcing, ore) ultrasonic forcing.

3. Vortical flow as in claim 1, wherein the respective fluids have sufficient velocity and adequate density difference, given consideration of their respective miscibility, viscosity, concentration or temperature differences to self-assemble and self-align thereby creating discrete lamellae where each fluid flows independently with respect to flow direction and velocity whose adjacency enables diffusional transfer to a second material selected from the group consisting of adjacent flowing fluid, stabilized flowing fluid, stationary fluid or an adjacent solid surface.

4. Vortical fluid flow as in claim 1 wherein the combination of geometry of the spiral channel and the fluid flow rate are selected to control one or more parameters selected from the group that consists of interfacial contact, rate or magnitude of diffusional mixing of adjacent flows such that at least one of the following effects selected from the group consisting of maximized reactivity, heat transfer, prevention of unwanted mixing, while facilitating axial flow to minimize the length of the interfacial contact needed for a process to come to completion.

5. Vortical fluid flow as in claim 1 where the minimal density difference needed for lamellar formation is 0.02 g/ml of miscible fluids.

6. Vortical fluid flow as in claim 1 where the minimal density difference needed for lamellar formation is 0.001 g/ml of miscible fluids.

7. Vortical flow as in claim 1 wherein one or more fluids comprise an interface between adjacent fluids to act as a dynamic membrane with selective permeation properties.

8. Vortical flow of two or more fluids as in claim 1 wherein density differences are employed to allow selective buoyancy of materials from the group consisting of microsolids, particles, filaments or cells to enable controlled location in a given lamella or at the interface between adjacent lamellae.

9. An apparatus that supports vortical flow as in claim 1 whose fluid is driven by centrifugal rotation of a body consisting of at least one spiral channel configured such that a rotational axis can accept any orientation and can operate in any gravitational field wherein gravitational forces act to limit the height of a vertically oriented array.

10. An apparatus that supports vortical flow as in claim 9 wherein a multiplicity of channels enables a multiplicity of reactions to occur simultaneously or sequentially such that some or all of the reactions that constitute a process sequence that involves at least one fluid and one fluid or one solid surface and where fluids may be added to or subtracted from a given channel where the product of a first reaction may become a reactant in a next reaction and that such plurality of reactions can occur in a single apparatus.

11. An apparatus as in claim 9 comprising a rotating spiral with a multiplicity of independent fluid inputs to each of a plurality of channels wherein the channel containing rotor rotates independent of the housing and wherein a heavy phase input and a light phase output are provided discrete fluid flow ports and the heavy phase output and the light phase input enter or exit the spiral from discrete ports in the stationary housing.

12. An apparatus as in claim 9 wherein a liquid pump is applied to a spiral channel apparatus in which at least one fluid is a gas and where the liquid is at its boiling point such that the pump provides the pressure needed to drive the liquid to the central rotational axis for it to exit where the exit pressure may not be less than the vapor pressure of the liquid without disrupting pressure balance of the system without the need for cooling or condensation thus maintaining the desired system pressure balance and thus assuring a net positive pressure head for the dense phase liquid throughout the apparatus.

13. Vortical flow in a spiral channel as in claim 1 defined as a limited access bounded space, rectilinear or curvilinear in cross-section, where the length greatly exceeds height or width, where its geometry conforms to any of the following arithmetic formulae: Archimedean where r=a+b*θ, equiforce where r=a/cos α, or MacInnes where r tan α=a, the involute of a circle where r=a/cos α or a Fermat spiral where r=θ̂0.5 provided that flow in the spiral channel enables constant along the axis of flow and the radial axis.

14. An apparatus as in claim 9 having a set of spiral channels wherein a multiplicity of spiral channels are organized into a platen or rotor to allow discrete zones in physical alignment, where one or more platens can be linked in serial or parallel array.

15. An apparatus as in claim 9 where the number of liquid lamellae radially aligned sum to give the width of the channel and where sufficient width remains to accept any gas where commonly the width of each lamella (discrete fluid flow) in a channel is on the order of 50-5000 μm and the number of lamellae serially aligned gives the width of the channel.

16. An apparatus as in claim 9 manufactured by machining, 3-D printing, stamping, molding, rolling or any other method known in the art and where the apparatus may be assembled into a unitary element by means of gluing, thermal or pressure welding, bolting, spring fit or any other assembly method known in the art.

17. Vortical flowing fluids as in claim 1 wherein one or more fluids flow and such fluids are members of the list that is comprised of a gas, a liquid or a flowable solids or any combination thereof where such fluids include but not limited to members of the following list: aqueous, biological, organic, ionic liquids, eutectic salts, protic and non-protic, magnetodynamic, sols, gels, suspensions, miscible and immiscible, where the fluid is a liquid, a sol, a gel, or contains suspended particulates, thixotropic fluids, Newtonian or non-Newtonian fluids.

18. Vortical flowing fluids as in claim 8 wherein buoyant particles, filaments, or beads participate in the reaction either directly or by bearing a catalyst or biocatalyst to promote interfacial reactions.

19. Vortical flowing fluids as in claim 1 that will support any of the following reactions or processes: absorption, adsorption, desorption, evaporation, distillation, desalination, extraction, redox, acid-base and group transfer reactions.

20. An apparatus as in claim 9 where exogenous energy may be delivered to any flow in a given lamella to promote one or more reactions at a given locale throughout the spiral or along the full length of the channel where the exogenous energy is in the form of centrifugal rotation, vibrational (acoustic or ultrasonic), magnetic, electromagnetic, photic, thermal or radiation energy or catalytic enhancement.

21. An apparatus as in claim 9 where energy may be delivered by means of electrodes, fibers, filaments, parallel flow channels or wires to a channel segment or a specific lamella at such time and place as may benefit a reaction or physicochemical process.

22. An apparatus as in claim 9 where endogenous energy may be collected and removed from any flow in a given lamella or sets of flows by virtue of electrodes, fibers, filaments, parallel flow channels or wires in the form of vibrational (acoustic or ultrasonic), electromagnetic, photic, thermal or radiation energy and can be delivered to exogenous sites for capture, storage or use.

23. An apparatus as in claim 9 where sensors embedded into or adjacent to or part of the spiral can detect signals by any means necessary, including but not limited to optical, vibrations, electromagnetic, thermal, pH, that can be used in a feedback or feed forward loop to control and preferably optimize reaction parameters in part by altering flow rates and any other controlling variable such a rotational rate in centrifugally rotating fluid.

24. An apparatus containing a series of spiral channels, rotating or stationary, wherein each channel is separated from the adjacent channel by means of a membrane, a membrane being defined as an ultrathin sheet whose length and width as significantly greater than its height.

25. An apparatus as in claim 25 where the separating membrane that exhibits selective properties e.g., particle size, proton or electron charge, ionic characteristics or physicochemical state that allow specific and defined materials to cross the membrane far more readily than do other components of the mixture and thereby allow two adjacent fluids of relatively similar density to react without mixing.
